(19) United States
(12) Patent Application Publication
(10) Pub. No.: US 2026/0278799 A1
(43) Pub. Date: Sep. 17, 2026

(54) METHOD FOR MONITORING HEALTH, EXTENDED REALITY DEVICE AND STORAGE MEDIUM

(71) Applicants: Fu Tai Hua Industry (Shenzhen) Co., Ltd., Shenzhen (CA); HON HAI PRECISION INDUSTRY CO., LTD., New Taipei (TW)

(72) Inventor: SHU-HAO LIU, New Taipei (TW)

(21) Appl. No.: 19/536,607

(22) Filed: Feb. 11, 2026

(30) Foreign Application Priority Data
Mar. 11, 2025 (CN) 202510285831.6

(51) Int. Cl. G06T 7/00; A61B 5/00; A61B 5/01; A61B 5/024; A61B 5/11; G06T 7/73

(52) U.S. Cl. CPC G06T 7/0016; A61B 5/0077; A61B 5/01; A61B 5/02416; A61B 5/1116; G06T 7/74; G06T 2207/10016; G06T 2207/10024; G06T 2207/10048; G06T 2207/30004; G06T 2207/30201

(57) ABSTRACT

A method for monitoring health, an extended reality device, and a storage medium are disclosed in the present application. The method includes: obtaining a first target video captured by the RGB camera and a second target video captured by the infrared camera; obtaining at least one set of first target data based on the first target video, obtaining at least one set of second target data based on the second target video, each set of second target data comprising a second face region of interest, corresponding second physiological parameter, and environmental temperature; determining whether abnormal physiological parameter is generated based on the human posture data and the environmental temperature within each dataset; and marking a corresponding face region of interest and outputting a prompt message when the abnormal physiological parameter is determined.

### CROSS-REFERENCE TO RELATED APPLICATIONS

The present application claims the benefit of and priority to Chinese patent application NO. 202510285831.6, field on Mar. 11, 2025, in China National Intellectual Property Administration, the entirety of which is incorporated herein by reference.

### FIELD

The present application relates to the field of extended reality technology, and in particular relates to a method for monitoring health, an extended reality device and a storage medium.

### BACKGROUND

In the field of extended reality technology, the extended reality devices may combine the real-world scenes with virtual scenes to construct a human-machine interactive virtual space. With the development of extended reality technology, these devices will increasingly be deployed for monitoring in multi-person settings such as airports, hospital wards, rehabilitation centers, stadiums, schools, and corporate facilities. In such scenarios, the ability to effectively identify individuals exhibiting health issues and provide early prevention or attention can significantly reduce the occurrence of unexpected incidents.

Therefore, improvement is desired.

### BRIEF DESCRIPTION OF THE DRAWINGS

To more clearly illustrate the technical solutions in the embodiments of the present application, the accompanying drawings used in the description of the embodiments will be briefly introduced below. The accompanying drawings described below are only some embodiments of the present application, for those skilled in the art, other drawings may be obtained based on these drawings without creative effort.

FIG. 1 is a schematic diagram of an extended reality device of an embodiment of the present application.

FIG. 2 is a flowchart of a method for monitoring health of an embodiment of the present application.

FIG. 3 is a flowchart of a process before executing step S206 in the flowchart shown in FIG. 2 provided in an embodiment of the present application.

FIG. 4 is a flowchart of sub steps of step S206 provided by an embodiment of the present application.

FIG. 5 shows a curve graph illustrating how human body temperature changes with environmental temperature in one embodiment of the present application.

FIG. 6 is a block diagram illustrating a storage medium of an embodiment of the present application.

### DETAILED DESCRIPTION

The following will describe the technical solution of the present application clearly and completely in combination with the accompanying drawings in the embodiments of the present application. The described embodiments are only part of the embodiments of the present application, not all of them. All other embodiments obtained by ordinary technicians in the art without making creative efforts based on the embodiments of the present application fall within the protection scope of the present application.

The following will provide a detailed explanation of some embodiments of the present application in conjunction with the accompanying drawings. In the absence of conflicts, the following embodiments and the features within them may be combined with each other.

The embodiments of the present application provide a method for testing semiconductor and an operation device. The present application may solve the problem of inaccurate testing and costly caused by using a full functional CPU in the process of testing the quality of the semiconductor pins.

In general, the extended reality devices may combine the real-world scenes with virtual scenes to construct a human-machine interactive virtual space. With the development of extended reality technology, these devices will increasingly be deployed for monitoring in multi-person settings such as airports, hospital wards, rehabilitation centers, stadiums, schools, and corporate facilities. In such scenarios, the ability to effectively identify individuals exhibiting health abnormalities and provide early prevention or attention can significantly reduce the occurrence of unexpected incidents, therefore, how to monitor the health of the population in the above-mentioned places in real time has become an urgent problem to be solved.

In order to solve the above problems, the present application provides a method for monitoring health, a storage medium, and an extended reality device that may balance both the efficiency and accuracy of health monitoring.

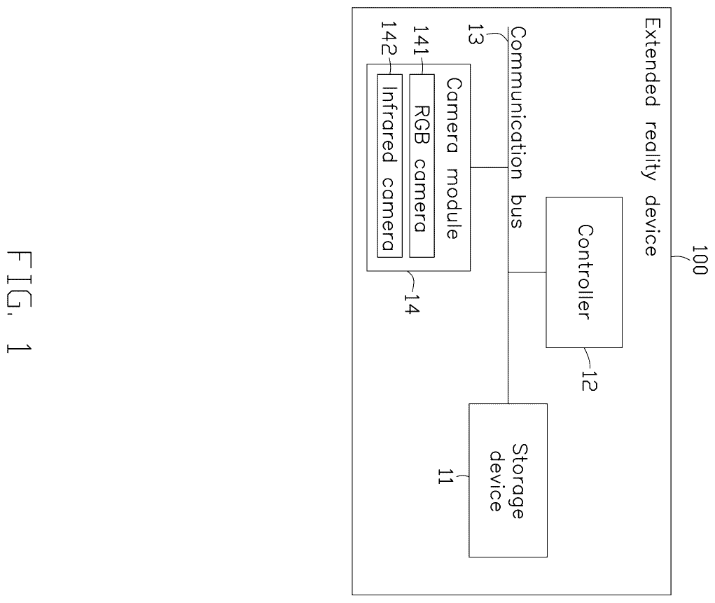

FIG. 1 illustrates an extended reality device 100 in accordance with an embodiment of the present disclosure.

The method for monitoring health provided by the embodiments of the present application is applied to the extended reality device 100. For example, the method may be executed by a controller configured within the extended reality device 100. The extended reality device 100 includes, but is not limited to, a storage device 11, at least one controller 12, and a camera module 14 interconnected via a communication bus 13. The camera module 14 may be a camera device installed on the extended reality device 100, such as an RGB camera 141 and an infrared camera 142.

FIG. 1 merely illustrates one example of the extended reality device 100 and does not constitute a limitation on the extended reality device 100. In actual applications, the extended reality device 100 may include more or fewer components shown in the diagram, combine certain components, or replace different components. For example, the extended reality device 100 may also include input/output devices, network access devices, and display screens.

In the embodiments of the present application, the extended reality device 100 may be a wearable device. For example, in some application scenarios of the present application, the extended reality device 100 may be a smart helmet or a smart glass.

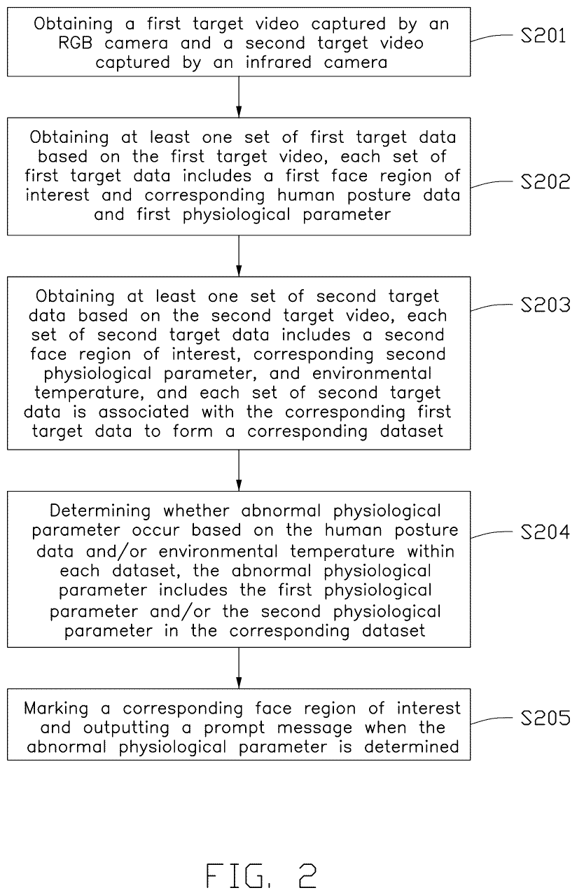

FIG. 2 is a flowchart of a method for monitoring health of an embodiment of the present application. It is understood that the order of steps in the flowchart may be altered and certain steps may be omitted based on different requirements. In some application scenarios of the present application, the steps shown in FIG. 2 may be performed by the controller 12.

As shown in FIG. 2, the method for monitoring health may include the following steps:

At step S201, obtaining a first target video captured by an RGB camera and a second target video captured by an infrared camera.

In some embodiments, before executing step S201, calibration of the intrinsic parameters and extrinsic parameters of the RGB camera 141 and the infrared camera 142 may be performed using a calibration pattern board, and the images in the first target video and the second target video obtained by the RGB camera 141 and the infrared camera 142 for image acquisition of the same target scene are aligned to facilitate subsequent image processing.

At step S202, obtaining at least one set of first target data based on the first target video. Each set of first target data includes a first face region of interest and corresponding human posture data and first physiological parameter.

The present application may obtain human images from the first target video using object detection algorithms and Kalman filter algorithms, it then extracts and tracks the skeleton data for each human image based on a preset human posture estimation algorithm, and extracts human posture data from the skeleton data of each human image. The human posture data may include the skeletal point information of each human image. In some embodiments, the human posture estimation algorithm may be at least one of the OpenPose algorithm, the DensePose algorithm, or other similar algorithms.

The first physiological parameter includes at least one of heart rate data and blood pressure data. It should be understood that heart rate refers to the number of heart beats per minute in a normal person at rest. In the present application, the heart rate data may be obtained through remote photoplethysmography (rPPG) technology. The pulse cycle causes changes in blood volume in blood vessels over time. The blood volume in blood vessels affects the absorption of red, blue and green light to varying degrees, by extracting the signal strength of red, blue and green light captured by the camera, the heart rate may be calculated. For example, in step S202, based on the trained face recognition model, the first face region of interest in each frame of the first target video within the preset time period may be extracted, and then the images of the forehead and cheek regions in each first face region of interest may be extracted. Then, the R-pixel mean value, G-pixel mean value, and B-pixel mean value of the forehead and cheek regions corresponding to each first face region of interest in each frame of the image may be calculated, and the pixel mean may be calculated, based on the frame rate of the RGB camera 141, the time required between two consecutive peaks of the pixel mean value may be calculated to convert it into the heart rate. In some embodiments, the average heart rate may be calculated every 20 seconds based on the above algorithm.

In some embodiments, the blood pressure data corresponding to each first face region of interest may be calculated based on a deep learning network architecture, using the computed pixel mean value input for each frame. For example, feature data such as wave peaks (maximum values) and wave troughs (minimum values), rise time (time from trough to peak), fall time (time from peak to next trough), peak width (width at the peak), and trough width may be obtained based on the pixel mean value of each frame, and this feature data is then input into the deep learning network to extract information about vascular elasticity and blood flow characteristics, which is subsequently used for blood pressure estimation. The present application does not limit the deep learning network architecture model used in step S202.

In step S202, the overlapping pixels between each first face region of interest and each human image may be calculated sequentially, and the human image with the most overlapping pixels is selected as a target human image from all human images. Then, a mapping relationship may be established between each first face region of interest and the corresponding human posture data of the target human image, thereby associating the human posture data with the corresponding first face region of interest to form a set of first target data. The number of first target data may be equal to the number of humans appearing in the first target video, in other words, the first target data corresponding to each human in the first target video may be established based on the first target video.

At step S203, obtaining at least one set of second target data based on the second target video, each set of second target data includes a second face region of interest, corresponding second physiological parameter, and environmental temperature, and each set of second target data is associated with the corresponding first target data to form a corresponding dataset.

The second target video may be an infrared image formed by infrared energy emitted from a temperature of the object. Furthermore, since each pixel value in an infrared image correlates with the intensity of infrared radiation emitted from a surface of the object, and since radiation intensity is directly related to the temperature of the object, the temperature of the corresponding target in the image may be directly calculated based on each pixel value.

The second face region of interest refers to the face region extracted from each frame within a preset time interval of the second target video. The second physiological parameter includes at least one of human body temperature data and c rate data, and respiration rate denotes the number of breaths per minute. In some embodiments, based on the extracted forehead and cheek region images corresponding to each second face region of interest, the temperature mean value calculated from pixels corresponding to the forehead and cheek regions of each extracted second face region of interest may be computed as human body temperature data. Then, based on the frame rate of the RGB camera 141, the time required between two consecutive peaks of the temperature mean value may be calculated to convert it into the respiration rate. In some embodiments, the average respiration rate may be calculated every 20 seconds based on the above algorithm. In other embodiments, image correction and filtering may first be performed on either the first target video or the second target video, the temperature value of each pixel in the second target video is mapped to the corresponding pixel in the first target video to generate a third target video. Subsequently, the second face region of interest is obtained within the third target video based on the first face region of interest, and corresponding second physiological parameter may be calculated using the forehead and cheek region images within the second face region of interest and the aforementioned algorithm. This approach enables the acquisition of more precise second physiological parameters even when the face is partially obscured by a mask.

In some embodiments, in step S203, the human image and background region of interest may be extracted from the second target video. The background region of interest may be any pixel region in the second target video other than the human image. The temperature mean value is calculated based on the temperature values of each pixel in the background region of interest to obtain the environmental temperature. In other embodiments, the environmental temperature may also be calculated based on other algorithms applied to the second target video; this application does not impose restrictions on specific algorithms.

Since the first target video and the second target video are obtained by image acquisition of the same target scene, in step S202, image feature matching may also be performed based on the first face region of interest and the second face region of interest, so that when the first face region of interest and the second face region of interest match each other, by correlating the first face region of interest and the second face region of interest of the same target human image, the first physiological parameter, the second physiological parameter, the environmental temperature and the human posture data of the same target human image may be associated, and each set of second target data is associated with the corresponding set of first target data to form a dataset. Therefore, health monitoring of the associated images of the same target human may be conducted based on the first target data and the second target data. The number of datasets may be equal to the number of humans appearing in the first target video or the second target video, several datasets corresponding to each human may be established based on the first target video or the second target video.

At step S204, determining whether abnormal physiological parameter occur based on the human posture data and/or environmental temperature within each dataset, the abnormal physiological parameter includes the first physiological parameter and/or the second physiological parameter in the corresponding dataset.

In step S204, the state of each human image may be determined based on the human posture data within a preset time period, such as the motion state or non-motion state. The non-motion state may be a sitting state or a stationary standing state. The motion type during movement may be further determined based on human posture data. For example, the human posture data in each dataset may be compared with each preset posture data in the motion database to determine the preset posture data with the highest similarity. The preset posture data is associated with the corresponding motion type. Thus, the human posture data in each dataset may determine the corresponding motion type based on the most similar preset posture data.

The corresponding exercise intensity varies under different states or types of exercise. Moreover, due to the influence of exercise intensity and environmental temperature, the first physiological parameter and the second physiological parameter may differ for the same individual under varying exercise intensities and/or temperature temperatures. Thus, in step S204, the corresponding motion type and/or physiological parameter range for the given environmental temperature may be determined based on the human posture data and/or environmental temperature, determine whether abnormal physiological parameters occur based on whether the first physiological parameter and/or the second physiological parameter meet the corresponding physiological parameter range. Therefore, the method for monitoring health provided in the present application may improve the accuracy of health monitoring.

At step S205, marking a corresponding face region of interest and outputting a prompt message when the abnormal physiological parameter is determined.

For example, the first target video may be displayed on the screen of the extended reality device 100, highlighting the first face region of interest corresponding to the abnormal physiological parameter in the first target video and outputting the prompt message. Therefore, when users of the extended reality device 100 may intervene in a timely manner to prevent unexpected events from occurring when they see the prompt message.

In summary, the method for monitoring health provided by the present application obtains the first target video and the second target video respectively captured by the RGB camera 141 and infrared camera 142, obtains the first face region of interest and corresponding human posture data and the first physiological parameter through the first target video, obtains the environmental temperature, the second face region of interest, and second physiological parameter through the second target video, determines whether abnormal physiological parameters exist based on the human posture data and/or environmental temperature, that is, whether the first physiological parameter and the second physiological parameter are abnormal data. Thus, the method for monitoring health provided herein validates the first physiological parameter and the second physiological parameter using the human posture data and the environmental temperature, this may reduce detection errors caused by monitoring only the first physiological parameter and the second physiological parameter in related technologies, thereby enhancing the accuracy of health monitoring. As the method for monitoring health provided in the present application may be applied in real time to the extended reality device 100, it effectively improves the efficiency of health monitoring.

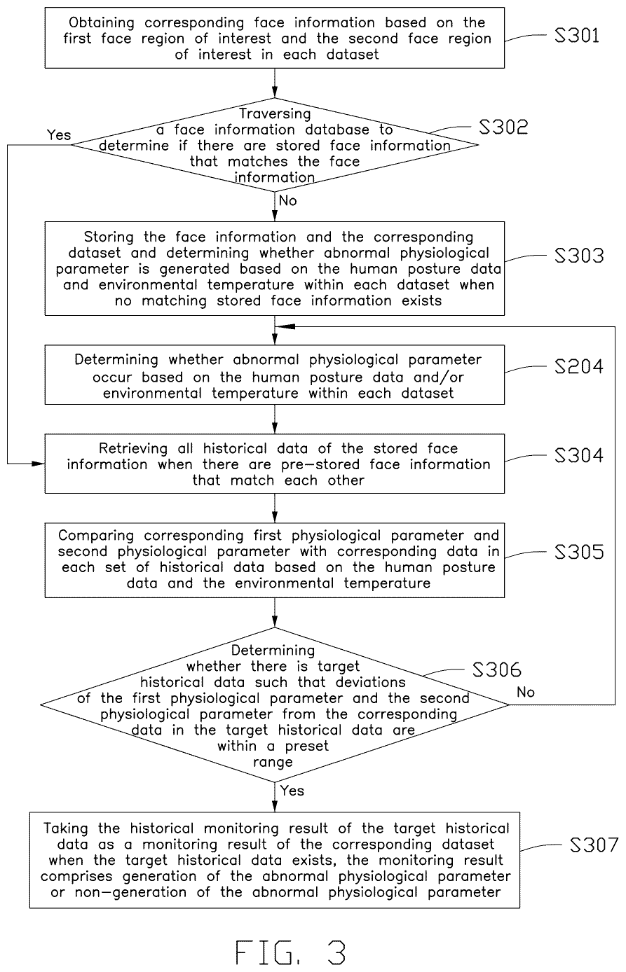

Referring to FIG. 3, in some embodiments, before executing step S204, the method further includes:

At step S301, obtaining corresponding face information based on the first face region of interest and the second face region of interest in each dataset.

The face information may be feature vectors extracted based on the first face region of interest and the second face region of interest, such as feature vectors related to face characteristics including face contours, eyebrow positions, interpupillary distance, skin texture, and pore distribution, this application does not impose restrictions on the algorithm used to obtain face information in step S301. For example, in some embodiments, the face information of the face regions of interest may be extracted based on algorithms such as convolutional neural networks, LBP (Local Binary Patterns), HOG (Directional Gradient Histogram), etc.

At step S302, checking a face information database to determine if there are stored face information that matches the face information.

The face information database stores multiple pre stored face information and associated historical data, such as name, age, and historical health monitoring data. In step S302, the face information database is traversed to sequentially compare the face information corresponding to at least one first face region of interest extracted in step S301 with the pre-stored face information in the database, this process calculates the similarity or matching feature vector between the face information and the pre-stored face information. When the face database contains pre-stored face information matching the face information in step S301 with a similarity reaching the preset threshold, or contains pre-stored face information with matching feature vectors reaching the preset value, it is determined that pre-stored face information exists that mutually matches the face information. Accordingly, when the similarity between the stored face information in the face information database and the face information extracted in step S301 is less than a preset similarity threshold, or when the number of matching feature vectors for the stored face information in the database is less than a preset value, it is determined that no stored face information in the database matches the given face information.

At step S303, storing the face information and the corresponding dataset and determining whether abnormal physiological parameter is generated based on the human posture data and environmental temperature within each dataset when no matching stored face information exists. After executing step S303, the controller proceeds to execute the steps S204 and S205

and stores the monitoring result corresponding to the face information. The monitoring result includes generation of the abnormal physiological parameter or non-generation of the abnormal physiological parameter. When the abnormal physiological parameter is generated, the corresponding abnormal physiological parameter is also marked within the stored information.

At step S304, retrieving all historical data of the stored face information when there are pre-stored face information that match each other.

Each set of historical data may include at least the first historical physiological parameter, historical human motion posture data, second historical physiological parameter, historical environmental temperature, and historical monitoring result. The historical data may be the first physiological parameter, second physiological parameter, environmental temperature, human posture data, and monitoring result corresponding to face information captured in the past time. In some embodiments, the historical data may also be data retrieved from other databases, such as cloud servers. The human posture data may reflect the corresponding motion state of the corresponding human. Therefore, through each set of historical data, the physiological parameters of the corresponding person in a certain motion state in the past and/or at a certain environmental temperature may be determined. For example, the heart rate range of a person during rest or during a certain exercise. These historical data may be applied in the future to determine whether the heart rate of the same person in the same exercise state falls within the historical data range, in order to improve the accuracy of identifying abnormal physiological parameter through personalized data.

At step S305, comparing corresponding first physiological parameter and second physiological parameter with corresponding data in each set of historical data based on the human posture data and the environmental temperature.

Since both environmental temperature and motion state may affect the first physiological parameter and the second physiological parameter, the corresponding physiological parameter ranges may be determined respectively by the environmental temperature and the motion state. Therefore, when the detection accuracy is relatively loose, the human posture data in the dataset may first be compared with the corresponding historical posture data in each set of historical data, or the environmental temperature in the dataset may be compared with the corresponding historical environmental temperature in each set of historical data. This way, when the human posture data matches the corresponding historical posture data or the environmental temperature matches the corresponding historical environmental temperature, the corresponding historical data is determined as the reference historical data to determine whether abnormal physiological parameters have occurred based on the first historical physiological parameter and the second historical physiological parameter in the reference historical data. When the detection accuracy requirements are relatively strict, the human posture data in the dataset may be compared with the corresponding historical posture data in each set of historical data, and the environmental temperature in the dataset may be compared with the corresponding historical environmental temperature in each set of historical data. In this way, when the human posture data matches the corresponding historical posture data and the environmental temperature matches the corresponding historical environmental temperature, the corresponding historical data is determined as reference historical data to determine whether abnormal physiological parameters have occurred based on the first physiological parameter and the second historical physiological parameter in the reference historical data.

The human posture data and the environmental temperature are respectively matched with the corresponding historical posture data and historical environmental temperature, which may be that the deviation of the human posture data from the corresponding historical posture data is within a first deviation range, and the deviation of the environmental temperature from the corresponding historical environmental temperature is within a second deviation range. For example, the first deviation range and the second deviation range may represent positive and negative deviation thresholds, such as ±k. The first deviation range may be ±k1, the second preset deviation range may be ±k2, and k1 and k2 may be any positive numerical values.

After determining the reference historical data, the first physiological parameter and the second physiological parameter of the same target human image may be compared with the first historical physiological parameter and the second historical physiological parameter in the reference historical data. In this way, in step S305, the reference historical data with practical value may be first screened out from the historical data of the same person through the human posture data and the environmental temperature, thereby improving the monitoring accuracy through individualized data.

At step S306, determining whether there is target historical data such that deviations of the first physiological parameter and the second physiological parameter from the corresponding data in the target historical data are within a preset range.

The preset range is used to represent the deviation range between the corresponding data. For example, the preset range may include ±k3 and ±k4, and k3 and k4 c may be any positive numerical values. For example, when the deviation between the first physiological parameter and the first historical physiological parameter is within the range of ±k3, and the deviation between the second physiological parameter and the second historical physiological parameter is within the range of ±k4, the reference historical data corresponding to the first historical physiological parameter and the second historical physiological parameter are determined as the target historical data. When only the first physiological parameter is obtained for a target human image and the deviation between the first physiological parameter and the first historical physiological parameter is within the range of +k1, the reference historical data corresponding to the first historical physiological parameter is determined as the target historical data. When only the second physiological parameter is obtained for a target human image and the deviation between the second physiological parameter and the second historical physiological parameter is within the range of ±k2, the reference historical data corresponding to the second historical physiological parameter is determined as the target historical data.

In some embodiments, when there are multiple sets of reference historical data such that the deviations between the first physiological parameter and the second physiological parameter and the corresponding first historical physiological parameter and second historical physiological parameter are within the preset range, the set of reference historical data with a smallest absolute deviation is selected as the target historical data.

At step S307, taking the historical monitoring result of the target historical data as a monitoring result of the corresponding dataset when the target historical data exists, the monitoring result comprises generation of the abnormal physiological parameter or non-generation of the abnormal physiological parameter.

When the target historical data exists, it refers to historical human posture data and historical environmental temperature that deviate from the human posture data and/or environmental temperature within the preset deviation range, and the first historical physiological parameter and the second historical physiological parameter that deviates from the first physiological parameter and the second physiological parameter within the preset range. This indicates that the currently detected physiological parameter conforms to the historical personal physiological state of the corresponding person, that is, the corresponding person has been in a similar condition to the current motion state and/or environmental temperature. Therefore, the monitoring result of the target historical data may be used as the current monitoring result to reduce algorithm complexity and save computing power. After executing step S307, the process may return to step S201 to retrieve the first target video and the second target video again.

When the result of step S306 is negative, it indicates that the reference historical data has not been determined, or the target historical data has not been determined after determining the reference historical data. At this point, the process should return to step S204.

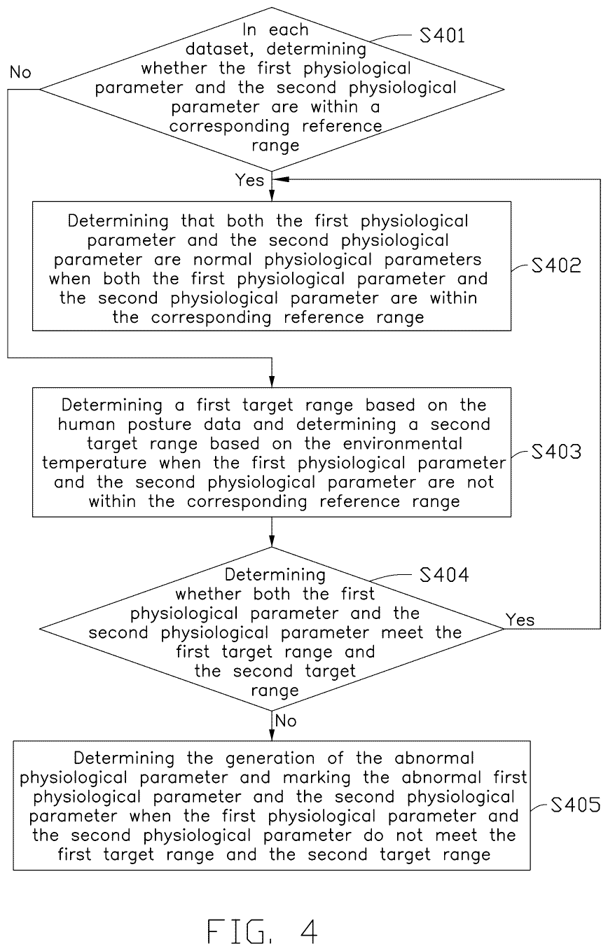

Referring to FIG. 4, in some embodiments, step S204 may include the following steps:

At step S401, in each dataset, determining whether the first physiological parameter and the second physiological parameter are within a corresponding reference range.

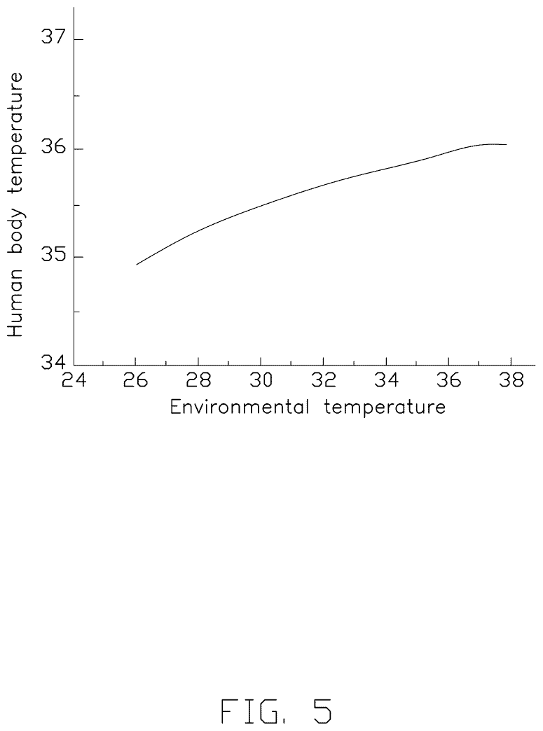

The reference range is used to indicate the range defined by the upper and lower limits of physiological parameters of the human body in a non-motion state. Furthermore, to enhance the monitoring accuracy, the corresponding reference range may also be determined based on at least one of the parameters such as environmental temperature, age, and gender. For example, please refer to FIG. 5, in some embodiments, the human temperature data and the environmental temperature data have a positive correlation as shown by the curve in FIG. 5. For instance, when the environmental temperature is 26° C., the normal range of human body temperature is 34.8° C. (±0.4° C.), when the environmental temperature is 38° C., the normal range of human body temperature is 36.0° C. (±0.4° C.), and the specific data may be obtained by measuring human body temperature under different environmental temperatures.

In some embodiments, the age of the corresponding human image may be determined based on the first target video and the first face region of interest, and then the reference range may be determined based on information such as environmental temperature and age. In some embodiments, a reference range lookup table may be preset in the storage device 11 (as show in FIG. 1). Thus, the controller 12 may perform a lookup operation based on the environmental temperature and age to determine the corresponding reference range. The reference range may include the first reference range and the second reference range. The first reference range is used to represent the upper and lower limits of the first physiological parameter of the human body in a non-motion state, and the second reference range is used to represent the upper and lower limits of the second physiological parameter of the human body in a non-motion state.

Taking the first physiological parameter as heart rate data and the second physiological parameter includes respiratory rate and body temperature as examples, when the human body is in a non-motion state and the environmental temperature is within the range of 26° C. to 38° C., the body temperature is between 34.8° C. (±0.4° C.) and 36.0° C. (±0.4° C.), and the respiratory rate is between 12 and 25 per minute., For elderly people aged 60 and above, or children aged 6 and under, the upper limit of respiratory rate may be increased to 40/minute; the heart rate is between 60-100/minute, and for children under 6 years old, the heart rate may be between 70-120/minute.

Therefore, in the non-motion state (such as lying down, standing, sitting still, sleeping, etc.), the corresponding reference range may be determined based on parameters such as environmental temperature and age to determine whether the abnormal physiological parameter is generated.

At step S402, determining that both the first physiological parameter and the second physiological parameter are normal physiological parameters when both the first physiological parameter and the second physiological parameter are within the corresponding reference range.

At step S403, determining a first target range based on the human posture data and determining a second target range based on the environmental temperature when the first physiological parameter and the second physiological parameter are not within the corresponding reference range.

The first target range may be the upper and lower limits of the first physiological parameter and the upper and lower limits of the second physiological parameter determined based on human posture data under the corresponding motion type. The second target range may be the upper and lower limits of the first physiological parameter and the upper and lower limits of the second physiological parameter determined based on the environmental temperature.

In step S403, the human posture data may be analyzed and processed based on the neural network model to output the corresponding motion type, the present application does not limit the prediction algorithm and neural network model adopted. In the present application, the storage device 11 may be preset with multiple motion type-first target range query tables. Thus, after the controller 12 determines the corresponding motion type based on the human posture data, it may perform a table lookup operation to determine the corresponding first target range. In some embodiments, to enhance detection accuracy, the age range of the corresponding human image may also be determined based on the first face region of interest and the second face region of interest in the dataset, so as to determine a more precise first target range according to the motion type and age range.

Similarly, multiple environmental temperature-second target range query tables may also be set in the storage device 11. Thus, controller 12 may perform table lookup operations based on the environmental temperature to determine the corresponding second target range. Taking the first physiological parameter as heart rate data and the second physiological parameter includes respiratory rate and body temperature data as examples, when in the motion state and the environmental temperature is within the range of 26° C. to 38° C., the determined second target range may be a respiratory rate of 40-70 per minute. The heart rate should be between 60 and 180 per minute. For children under 6 years old, the heart rate may be between 60 and 200 per minute.

In other embodiments, when the monitored object recovers from a motion state to a non-motion state, a preset duration, such as 20 minutes, is also required to restore the body temperature from the motion state to the non-motion state. Therefore, when it is confirmed that the duration for the monitored subject to recover from a motion state to a non-motion state is less than the preset duration, the first physiological parameter and the second physiological parameter may be tested according to the first target range and the second target range.

In other embodiments, the motion may also be classified based on the intensity, frequency and time of the motion type, so that multiple motion types may determine the first target range according to the query table of the same level, thereby reducing the complexity of the algorithm, the consumption of computing power and the occupation of memory. For example, the motions may be classified into three levels based on the intensity of the motion. The first level motion items may include intense physical activities such as race walking, jogging, running, rope skipping, cycling, aerobics, dancing, basketball, football and martial arts, etc. The second level motion items may include moderate intensity physical activities, such as brisk walking and throwing activities, etc. The third level items may include light exercises such as slow walking and strolling. Furthermore, the first target range corresponding to the level of motion items may be configured.

In other embodiments, the reference range, the first target range and the second target range may also be switched actively by the user. For example, when the user confirms that the current scene is a non-motion scene (such as a hospital ward, rehabilitation center, etc.), the user may switch to the reference range to verify the first physiological parameter and the second physiological parameter. When the user confirms that the current scene includes a motion scene (such as a school playground, stadium, factory, etc.), the user may switch to the first target range to verify the first physiological parameter and the second physiological parameter.

At step S404, determining whether both the first physiological parameter and the second physiological parameter meet the first target range and the second target range.

At step S405, determining the generation of the abnormal physiological parameter and marking the abnormal first physiological parameter and the second physiological parameter when the first physiological parameter and the second physiological parameter do not meet the first target range and the second target range.

When at least one of the following situations occurs: the first physiological parameter does not conform to the first target range, the first physiological parameter does not conform to the second target range, the second physiological parameter does not conform to the first target range, or the second physiological parameter does not conform to the second target range, the abnormal physiological parameter may be determined.

When both the first physiological parameter and the second physiological parameter meet the first target range and the second target range, it is determined that the first physiological parameter and the second physiological parameter are normal physiological parameters, the monitoring result is determined that no abnormal physiological parameter is generated.

Therefore, by executing steps S401 to S405, the first physiological parameter and the second physiological parameter may be further detected according to different motion states and different environmental temperatures, so as to improve the accuracy of health monitoring.

In some embodiments, the health monitoring method may further include: updating historical data based on the first physiological parameter, human posture data, the second physiological parameter and environmental temperature.

After determining that the first physiological parameter and the second physiological parameter are abnormal physiological parameters, or after determining that both the first physiological parameter and the second physiological parameter are normal physiological parameters, the first physiological parameter, human posture data, the second physiological parameter and the environmental temperature are stored as a set of corresponding historical data. Therefore, the first physiological parameter, human posture data, second physiological parameter and environmental temperature obtained in the current execution cycle may be used as historical data to participate in the health monitoring of the next execution cycle.

Since historical data may be used as the first physiological parameter and the second physiological parameter in the target historical data verification dataset by executing steps S301 to S307, in order to reduce the error in determining the monitoring result through steps S301 to S307, each dataset with monitoring result obtained by executing step S204 may also be marked as a preparatory dataset. Moreover, the target historical data in step S306 needs to be determined from several preparatory datasets. Therefore, the expansion of errors during the execution of steps S301 to S307 may be avoided, and the accuracy of health monitoring during the execution of steps S301 to

S307 may be improved.

By adopting the method for monitoring health of the present application, users of the extended reality device may continuously monitor the physiological parameters of populations in specific locations, quickly identify abnormal populations, and provide early prevention or attention, making health monitoring convenient and efficient.

Referring to FIG. 1, the present application also provides an extended reality device 100, the extended reality device 100 includes an RGB camera 141, an infrared camera 142 and a controller 12. The controller 12 is used to perform the method for monitoring health described in any of the above embodiments.

The controller 12 may be a Central Processing Unit (CPU), or it may be other general-purpose controllers or Digital Signal processors (DSP), Application Specific Integrated Circuit (ASIC), Field-Programmable Gate Array (FPGA) or other programmable logic devices, discrete gates or transistor logic devices, discrete hardware components, etc. The general-purpose controller may be a microcontroller. The controller 12 is the control center of the extended reality device 100, connecting all parts of the entire electronic device through various interfaces and lines.

The storage device 11 may be used to store computer programs and/or modules/units. The controller 12 realizes various functions of the extended reality device 100 by running or executing computer programs and/or modules/units stored in the storage device 11, as well as by invoking data stored in storage device 11. The storage device 11 may mainly include the program storage area and the data storage area. The program storage area may store the operating system, applications required for at least one function (such as sound playback function, image playback function, etc.), etc. The data storage area may store data created based on the use of electronic devices, such as video data, audio data, phone books, etc. In addition, the storage device 11 may include high-speed random access memory, as well as non-volatile memory such as hard drives, memory, plug-in hard drives, smart media cards (SMC), secure digital (SD) cards, Flash Cards, at least one disk storage device, flash devices, or other volatile solid-state storage devices.

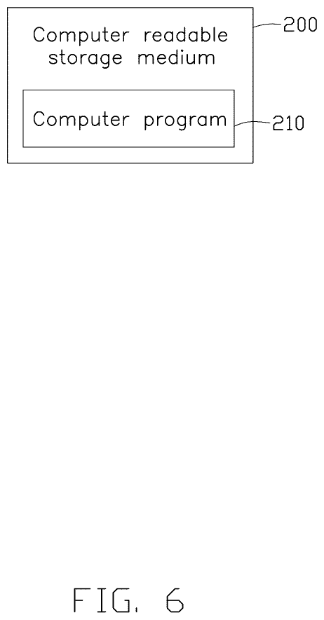

FIG. 6 illustrates a computer readable storage medium 200 in accordance with an embodiment of the present disclosure. The computer readable storage medium 200 stores a computer program 210 comprising at least one instruction, at least one instruction being executed by a controller in an electronic device to implement the method for monitoring health as described in any one embodiment.

For example, the computer program 210 may be divided into one or more modules/units, which are stored in the computer readable storage medium 200 and executed by the controller 12 to complete the present application. One or more modules/units may be a series of computer program instruction segments capable of performing specific functions, which are used to describe the execution process of computer programs in the electronic device.

The present application realizes all or part of the processes in the above-mentioned embodiments. It may also be accomplished by instructs the relevant hardware through a computer program. The computer program may be stored in a computer readable storage medium. When the computer program is executed by the controller, the steps of each of the above-mentioned embodiments can be realized. The computer programs include computer program codes, which may be in source code form, executable files, or certain intermediate forms, etc. The computer readable storage medium may include: any entity or device capable of carrying computer program code, recording medium, USB flash drive, portable hard disk, magnetic disk, optical disc, computer memory, read only memory (ROM), random access memory (RAM), electrical signals, and software distribution media, etc. It should be noted that the content included in computer readable storage medium may be appropriately increased or decreased in accordance with the requirements of legislation and patent practices within the jurisdiction. For example, in some jurisdictions, computer readable storage medium do not include carrier signals and telecommunication signals based on legislation and patent practices.

In the several embodiments provided in this application, it should be understood that the disclosed electronic devices and methods can be implemented in other ways. For example, the electronic device embodiments described above are only illustrative. For example, the division of modules is only a logical function division, and there may be other division methods in actual implementation.

In addition, the functional modules in various embodiments of the present application can be integrated into the same processing module, or each module can exist physically separately, or two or more modules can be integrated into the same module. The integrated modules mentioned above can be implemented in the form of hardware or in the form of a combination of hardware and software functional modules.

Even though numerous characteristics and advantages of the present technology have been set forth in the foregoing description, together with details of the structure and function of the present application, the application is illustrative only, and changes may be made in the detail, especially in matters of shape, size, and arrangement of the parts within the principles of the present application, up to and including the full extent established by the broad general meaning of the terms used in the claims. It will, therefore, be appreciated that the exemplary embodiments described above can be modified within the scope of the claims.

What is claimed is:

1. A method for monitoring health applied to an extended reality device, the extended reality device comprising an RGB camera and an infrared camera, the method comprising:
obtaining a first target video captured by the RGB camera and a second target video captured by the infrared camera;
obtaining at least one set of first target data based on the first target video, each set of first target data comprising a first face region of interest, human posture data corresponding to the first region of interest, and a first physiological parameter;
obtaining at least one set of second target data based on the second target video, each set of second target data comprising a second face region of interest, a second physiological parameter corresponding to the second face region of interest, and an environmental temperature, and associating each set of second target data with a corresponding first target data to form a corresponding dataset;
determining whether an abnormal physiological parameter is obtained based on the human posture data and the environmental temperature within each of the corresponding dataset, wherein the abnormal physiological parameter comprises the first physiological parameter and the second physiological parameter in the corresponding dataset; and
marking a corresponding face region of interest and outputting a prompt message in response to the abnormal physiological parameter been determined.

2. The method according to claim 1, before determining whether abnormal physiological parameter occur based on the human posture data and the environmental temperature, the method further comprising:
obtaining corresponding face information based on the first face region of interest and the second face region of interest in each dataset;
checking a face information database to determine whether the face information matches pre-stored face information; and
storing the face information and the corresponding dataset and determining whether abnormal physiological parameter is obtained based on the human posture data and the environmental temperature within each dataset in response to the face information does not match the prestored face information.

3. The method according to claim 2, further comprising:
retrieving all historical data of the pre-stored face information in response to the face information matches the prestored face information, wherein each set of historical data comprises:
first historical physiological parameter, historical human posture data, second historical physiological parameter, and historical environmental temperature;
comparing corresponding first physiological parameter and second physiological parameter with corresponding data in each set of historical data based on the human posture data and the environmental temperature; and
determining whether there is target historical data such that deviations of the first physiological parameter and the second physiological parameter from the corresponding data in the target historical data are within a preset range.

4. The method f according to claim 3, wherein the historical data comprises a historical monitoring result, the method further comprises:
taking the historical monitoring result of the target historical data as a monitoring result of the corresponding dataset in a case that the target historical data is included in the historical data, the monitoring result comprises a generation of the abnormal physiological parameter or a non-generation of the abnormal physiological parameter; and
determining whether the abnormal physiological parameter is generated based on the human posture data in each dataset and the environmental temperature in a case that the target historical data is not included in the historical data.

5. The method according to claim 1, wherein determining whether the abnormal physiological parameter is generated based on the human posture data and the environmental temperature within each of the corresponding dataset comprises:
in each dataset, determining whether the first physiological parameter and the second physiological parameter are within a corresponding reference range; and
determining both the first physiological parameter and the second physiological parameter are normal physiological parameters in a case that both the first physiological parameter and the second physiological parameter are within the corresponding reference range.

6. The method according to claim 5, further comprising:
determining a first target range based on the human posture data and determining a second target range based on the environmental temperature in a case that the first physiological parameter and the second physiological parameter are not within the corresponding reference range;
determining whether both the first physiological parameter and the second physiological parameter meet the first target range and the second target range; and
determining the generation of the abnormal physiological parameter and marking the abnormal first physiological parameter and the second physiological parameter in a case that the first physiological parameter and the second physiological parameter do not meet the first target range and the second target range.

7. The method according to claim 6, wherein the method further comprises:
determining the non-generation of the abnormal physiological parameter in a case that both the first physiological parameter and the second physiological parameter meet the first target range and the second target range.

8. The method according to claim 1, wherein the first physiological parameter comprises at least one of a heart rate and a blood pressure, and the second physiological parameter comprises at least one of a body temperature and a respiration rate.

9. An extended reality device comprising:
an RGB camera;
an infrared camera;
a storage device; and
at least one controller, wherein the storage device stores one or more programs, when executed by the at least one controller, the one or more programs cause the at least one controller to:
obtain a first target video captured by the RGB camera and a second target video captured by the infrared camera;
obtain at least one set of first target data based on the first target video, each set of first target data comprising a first face region of interest, human posture data corresponding to the first region of interest, and a first physiological parameter; and
obtain at least one set of second target data based on the second target video, each set of second target data comprising a second face region of interest, a second physiological parameter corresponding to the second face region of interest, and an environmental temperature, and associating each set of second target data with a corresponding first target data to form a corresponding dataset;
determine whether an abnormal physiological parameter is obtained based on the human posture data and the environmental temperature within each of the corresponding dataset, wherein the abnormal physiological parameter comprises the first physiological parameter and the second physiological parameter in the corresponding dataset; and
mark a corresponding face region of interest and outputting a prompt message in response to the abnormal physiological parameter been determined.

10. The extended reality device according to claim 9, wherein the at least one controller is further caused to:
obtain corresponding face information based on the first face region of interest and the second face region of interest in each dataset;
check a face information database to determine whether the face information matches pre-stored face information; and
store the face information and the corresponding dataset and determine whether abnormal physiological parameter is obtained based on the human posture data and the environmental temperature within each dataset in response to the stored face information does not match the prestored face information.

11. The extended reality device according to claim 10, wherein the at least one controller is further caused to:
retrieve all historical data of the pre-stored face information in response to the face information matches the prestored face information, wherein each set of historical data comprises: first historical physiological parameter, historical human posture data, second historical physiological parameter, and historical environmental temperature;
compare corresponding first physiological parameter and second physiological parameter with corresponding data in each set of historical data based on the human posture data and the environmental temperature;
determine whether there is target historical data such that deviations of the first physiological parameter and the second physiological parameter from the corresponding data in the target historical data are within a preset range.

12. The extended reality device according to claim 11, wherein the historical data comprises a historical monitoring result, wherein the at least one controller is further caused to:
take the historical monitoring result of the target historical data as a monitoring result of the corresponding dataset in a case that the target historical data is included in the historical data, wherein the monitoring result comprises a generation of the abnormal physiological parameter or a non-generation of the abnormal physiological parameter; and
determine whether the abnormal physiological parameter is generated based on the human posture data in each dataset and the environmental temperature in a case that the target historical data is not included in the historical data.

13. The extended reality device according to claim 9, wherein the at least one controller is further caused to:
in each dataset, determine whether the first physiological parameter and the second physiological parameter are within a corresponding reference range; and
determine both the first physiological parameter and the second physiological parameter are normal physiological parameters in a case that both the first physiological parameter and the second physiological parameter are within the corresponding reference range.

14. The extended reality device according to claim 13, wherein the at least one controller is further caused to:
determine a first target range based on the human posture data and determining a second target range based on the environmental temperature when the first physiological parameter and the second physiological parameter are not within the corresponding reference range;
determine whether both the first physiological parameter and the second physiological parameter meet the first target range and the second target range; and
determine the generation of the abnormal physiological parameter and mark the abnormal first physiological parameter and the second physiological parameter in a case that the first physiological parameter and the second physiological parameter do not meet the first target range and the second target range.

15. The extended reality device according to claim 14, wherein the at least one controller is further caused to:
determine the non-generation of the abnormal physiological parameter in a case that both the first physiological parameter and the second physiological parameter meet the first target range and the second target range.

16. A non-transitory storage medium having stored thereon instructions that, when executed by a controller of an extended reality device, causes the controller to perform a method for monitoring health, wherein the method comprises:
obtaining a first target video captured by the RGB camera and a second target video captured by the infrared camera;
obtaining at least one set of first target data based on the first target video, each set of first target data comprising a first face region of interest, human posture data corresponding to the first region of interest, and a first physiological parameter;
obtaining at least one set of second target data based on the second target video, each set of second target data comprising a second face region of interest, a second physiological parameter corresponding to the second face region of interest, and an environmental temperature, and associating each set of second target data with a corresponding first target data to form a corresponding dataset;
determining whether an abnormal physiological parameter is obtained based on the human posture data and the environmental temperature within each of the corresponding dataset, wherein the abnormal physiological parameter comprises the first physiological parameter and the second physiological parameter in the corresponding dataset; and
marking a corresponding face region of interest and outputting a prompt message in response to the abnormal physiological parameter been determined.

17. The non-transitory storage medium according to claim 16, wherein before determining whether abnormal physiological parameter occur based on the human posture data and the environmental temperature, the method further comprising:
obtaining corresponding face information based on the first face region of interest and the second face region of interest in each dataset;
checking a face information database to determine whether the face information matches pre-stored face information; and
storing the face information and the corresponding dataset and determining whether abnormal physiological parameter is obtained based on the human posture data and the environmental temperature within each dataset in response to the face information does not match the prestored face information.

18. The non-transitory storage medium according to claim 17, further comprising:
retrieving all historical data of the pre-stored face information in response to the face information matches the pre-stored face information, wherein each set of historical data comprises:
first historical physiological parameter, historical human posture data, second historical physiological parameter, and historical environmental temperature;
comparing corresponding first physiological parameter and second physiological parameter with corresponding data in each set of historical data based on the human posture data and the environmental temperature; and
determining whether there is target historical data such that deviations of the first physiological parameter and the second physiological parameter from the corresponding data in the target historical data are within a preset range.

19. The non-transitory storage medium according to claim 18, wherein the historical data comprises a historical monitoring result, the method further comprises:
taking the historical monitoring result of the target historical data as a monitoring result of the corresponding dataset in a case that the target historical data is included in the historical data, the monitoring result comprises a generation of the abnormal physiological parameter or a non-generation of the abnormal physiological parameter; and
determining whether the abnormal physiological parameter is generated based on the human posture data in each dataset and the environmental temperature in a case that the target historical data is not included in the historical data.

20. The non-transitory storage medium according to claim 16, wherein determining whether the abnormal physiological parameter is generated based on the human posture data and the environmental temperature within each of the corresponding dataset comprises:
in each dataset, determining whether the first physiological parameter and the second physiological parameter are within a corresponding reference range; and
determining both the first physiological parameter and the second physiological parameter are normal physiological parameters in a case that both the first physiological parameter and the second physiological parameter are within the corresponding reference range.
